(19) United States
(12) Patent Application Publication
(10) Pub. No.: US 2023/0213517 A1
(43) Pub. Date: Jul. 6, 2023

(54) METHOD FOR DETECTING VIRUS PARTICLES AND KITS THEREFOR

(71) Applicant: COVIRABIO GMBH, Wien (AT)

(72) Inventors: Hans LOIBNER, Wien (AT); Marko POGLITSCH, Wien (AT)

(21) Appl. No.: 18/000,509

(22) PCT Filed: Jun. 2, 2021
(86) PCT No.: PCT/EP2021/064857

§ 371 Date: Dec. 1, 2022

(30) Foreign Application Priority Data
Jun. 4, 2020 (AT) A 50486/2020

(51) Int. Cl. G01N 33/569; C12Q 1/70

(52) U.S. Cl. CPC G01N 33/56983; C12Q 1/701; G01N 2333/165; G01N 2333/948

(57) ABSTRACT

Disclosed is a method for detecting virus particles in a sample, comprising the steps of: (a) incubating the sample with at least one virus-binding molecule bound to a solid phase; and (b) detecting binding of virus particles to the at least one virus-binding molecule bound to the solid phase. Also disclosed is a kit for use in this method.

### FIELD OF THE INVENTION

The present invention concerns methods for detecting virus particles in a sample and kits therefor.

### BACKGROUND

In December 2019, a new coronavirus emerged in China causing acute respiratory disease and also affecting other organs, named coronavirus disease 2019 (COVID-19). The virus was identified to be a beta coronavirus related to severe acute respiratory syndrome coronavirus (SARS-CoV) and thus was named SARS-CoV-2. SARS-CoV-2 is the third known coronavirus to cross the species barrier in the last two decades. SARS-CoV-2 not only can cause severe and life-threatening respiratory infections (Acute Respiratory Distress Syndrome; ARDS) but also severe, life-threatening infections of many other tissues and organs in humans. Previously, SARS-CoV emerged in 2003 and Middle East respiratory syndrome coronavirus (MERS-CoV) in 2012. However, SARS-CoV-2 spread with unprecedented speed compared with these earlier coronaviruses, in large part because it is significantly more easily transmissible via aerosol. Because of the rapid increase in number of cases and uncontrolled and vast spread worldwide, the World Health Organization has declared SARS-CoV-2 a pandemic.

Due to this worldwide health crisis, there have been and still are manifold attempts worldwide to develop and provide validated diagnostics based on different and complementary methods for detection of virus-infected and infectious individuals as well as individuals that have developed SARS-CoV-2 specific antibodies as consequence of a resolved infection. Such assays are needed for patient contact tracing, identifying the viral reservoir hosts, and epidemiologic studies. Furthermore, the use of such assays is crucial to understand and to shape the lock-down in various countries for control of viral spread.

In addition, of course there are worldwide numerous R&D attempts to provide new or repurposed drugs that would positively influence the course of severe forms of COVID-19, as well as prophylactic vaccines based on different established and also experimental technologies to protect the population against infection. Also for these crucially important activities a set of validated diagnostics is needed as mentioned above for rational development.

In order to detect individuals infected with SARS-CoV-2 presently at least three assay types have been developed by a variety of academic institutions and companies: (1) nucleic acid-based tests, like the widely adopted RT-PCR assay, which detects the presence of the viral genome; (2) viral antigen tests, which detect the presence of viral proteins; and (3) serological or antibody tests, which detect the presence of serum antibodies specific for viral antigens.

Unrelated to virus detection, Ho et al (Antiviral Res. 2006 February;69(2):70-6) concerns the design and biological activities of novel inhibitory peptides for SARS-CoV spike protein and angiotensin-converting enzyme 2 interaction. Also unrelated to virus detection, Shang et al (Nature. 2020 May; 581(7807):221-224) relates to the structural basis of receptor recognition by SARS-CoV-2. Unrelated to SARS-CoV-2, WO 2011/057347 A1 discloses general methods for detecting an analyte (such as a microbe, a protein, a nucleic acid, a macromolecule, a small molecule, a drug or a virus) in a sample.

The measurement of the virus itself is mostly based on the real-time reverse transcriptase polymerase chain reaction (rRT-PCR) currently in use worldwide.

rRT-PCR Test:

The rRT-PCR test (real-time Reverse Transcription Polymerase Chain Reaction) amplifies and detects certain segments of the viral RNA specific for SARS-CoV-2. In this technique, a virus-specific ribonucleic acid (RNA) is rewritten into deoxyribonucleic acid (DNA) by a reverse transcriptase and amplified by the polymerase chain reaction (PCR). Results are generally visualized by real-time fluorescence. Commercial rRT-PCR tests for SARS-CoV-2 are available from various providers.

The test can be done on respiratory samples obtained by various methods, including a nasopharyngeal swab or sputum sample. Results are generally available within a few hours. However, the RT-PCR test performed with throat swabs is only reliable in the first week of the disease. Later on, the virus can disappear in the throat while it continues to multiply in the lungs. For infected people tested in the second week, alternatively sample material can then be taken from the deep airways by suction catheter, or coughed up material (sputum) can be used.

Commercial rRT-PCR tests are available from various providers, such as LabCorp, Quest Diagnostics, Mayo Clinic, Roche Diagnostics, Abbott Laboratories, Hologic, LabCorp, Thermo Fisher Scientific or Cepheid.

A variation of this detection of specific RNA based on RT-PCR is based on an isothermal nucleic acid amplification method. In March 2020, the FDA approved an automated assay based on this technology that offers certain advantages regarding speed.

Viral Antigen Test:

While RT-PCR tests look for RNA from the virus, viral antigen tests look for proteins from the surface of the virus. These are usually proteins from the surface spikes, and a nasal swab is used to collect samples from the nasal cavity. One of the difficulties has been finding a protein target unique to SARS-CoV-2. There are related coronaviruses that cause common cold.

Antigen tests are often seen as the only way it will be possible to scale up testing to the huge numbers that will finally be needed to detect acute infection. RT-PCR tests take time and trained personnel to run the tests. Isothermal nucleic acid amplification tests, such as the test from Abbot Labs, can only process one sample at a time per machine. An antigen test works by taking a nasal swab from a patient and exposing that to paper strips that contain artificial antibodies designed to bind to coronavirus antigens. Any antigens that are present will bind to the strips and give a visual readout in short time.

The problem is that in the case of respiratory viruses often there is not enough of the antigen material present in the nasal swab to be detectable. Unlike the RT-PCR test, which efficiently amplifies very small amounts of genetic material so that there is enough to detect, there is no amplification of viral proteins in an antigen test. In consequence, the sensitivity is limited and many of SARS-CoV-2 infected patients might be missed by such tests.

Antibody Tests:

To identify individuals who already were infected and have recovered, antibody tests have been established:

The basis of these antibody tests is the detection of the IgM/IgG and sometimes IgA antibodies, which the immune system of an infected individual produces in response to SARS-CoV-2. The detection of such antibodies is based on the well-established ELISA (enzyme-linked immunosorbent assay) method, according to which antibodies to antigens from a specific target are captured on a solid phase coated with one or more antigens. The antibodies captured on the solid phase are then detected with a secondary antibody capable of binding the captured antibodies via a simple colorimetric reporter assay. These tests are carried out either in central laboratories or as point-of-care tests In the United States, a first antibody test was approved by the FDA on in March 2020.

One advantage of antibody tests is that they are easier to use. Blood samples from skin punctures can be measured directly and do not have to be extracted first, as with RT-PCR. They are also cheaper than RT-PCR tests.

As there is a time lag between infection and antibody formation (40% of individuals have measurable serum antibodies after one week after infection, and 94% have measurable serum antibodies two weeks after infection), these tests are not suitable for determining whether an individual is SARS-CoV-2 positive, but whether an individual was already infected and thus does not represent a threat for spread of infection, regardless whether the individual during infection has developed symptoms or not. Antibody tests are particularly suitable for large-scale use to examine past infection rates in populations and can shed light on the extent to which a population is already infected in order to build up a possible herd immunity.

There is an urgent need for a virus detection method which addresses the various disadvantages mentioned above.

### SUMMARY OF THE INVENTION

The present invention provides a method for detecting virus particles in a sample, comprising the steps of: (a) incubating the sample with at least one virus-binding molecule bound to a solid phase; and (b) detecting binding of virus particles to the at least one virus-binding molecule bound to the solid phase.

The present invention also provides a kit for performing this method, comprising a manual, one or more solvents, one or more buffers, and/or one or more solid phases and/or one or more enzymes and/or one or more antibodies and/or one or more primers and/or one or more enzyme substrates and/or one or more inactivated virus or pseudo virus preparations.

Further, the present invention provides a kit for detecting virus particles in a sample, comprising:
a solid phase in particular a plate such as a 96-well plate or beads such as agarose beads or a solid phase having a graphene surface or a solid phase having a semiconducting surface,
a virus entry receptor for the virus particles bound to the solid phase, preferably wherein the virus entry receptor bound to the solid phase is enzymatically inactive,
preferably a soluble virus entry receptor for the virus particles, especially wherein the soluble virus entry receptor is enzymatically active,
optionally, at least one substrate for the enzymatic activity of the soluble virus entry receptor
optionally, at least one washing solution, and
optionally, at least one inactivated virus or pseudo virus preparations;

preferably wherein: the virus entry receptor bound to the solid phase is enzymatically inactive ACE2, the soluble virus entry receptor is soluble enzymatically active ACE2, preferably wherein the virus particles are SARS-CoV-1 particles, SARS-CoV-2 particles or HCoV-NL63 particles, especially SARS-CoV-2 particles.

### DETAILED DESCRIPTION OF THE INVENTION

The invention provides novel diagnostic approaches for the detection of SARS-CoV-2 and also other viruses. The described approaches clearly distinguish themselves from existing detection methods and offer, amongst other features, significant advantages with regard to biological relevance, simplicity of use and speed of analysis (point-of-care). The innovative approach of a “virus entry receptor binding test” (VERB test) not only can be applied to identify SARS-CoV-2, but also represents a platform technology that can be applied also for detection of other viruses.

In the context of the current SARS-CoV-2 pandemic, it is assumed that the virus will still be a threat for quite some time to come. Slightly declining numbers of individuals with new infection in some parts of the world may indicate that the peak of the wave of infections has been overcome. Nevertheless, still only a very small part of the population has already been infected. The fear that the corona pandemic could flare up again after various stringent measures against exponential spread have been relaxed again is therefore highly relevant. Effective rapid tests are an important weapon in the fight against the continuing spread of COVID-19, and extensive and rapid test modalities are a key to contain the pandemic without economic collapse.

Like many other viruses, coronaviruses are also genetically variable, which means that their RNA sequence and their surface structures can change. As a result of these changes, existing test systems may no longer work, which was for example observed between SARS-CoV-1 and SARS-CoV-2. For this reason, when SARS-CoV-2 appeared, tests for the specific detection of this virus had to be developed, which cost valuable time at the beginning of a dangerous and rapid spread. The important control of such a spread of course relies on accurate and specific tests.

To date, the classical plaque assay is the only known way to gain information on the functionality of a virus particle, e.g. its infectious potential. Due to the fact that infectious virus particles are enriched during the assay, it has to be performed in a cell culture laboratory, e.g. for SARS-CoV-2 at biosafety level 3 (BSL-3). This is only one of the factors greatly limiting its applicability in routine testing. In order to perform a plaque assay for SARS-CoV-2 or other respiratory viruses, typically lung cell lines are cultivated in the presence of different dilutions of the sample of interest for several days. During this incubation period, infectious virus particles within the sample infect the target cells, replicate within these cells and infect the adjacent cells, leading to patches of destroyed cells in the cell layer, which are finally stained before counting. The so-called cytopathic effect observed is a product of full virus functionality, investigated in a cell type or cell line mimicking the in vivo cellular target of a virus, being a very close to in vivo model of evaluating the infectivity of a biological sample. The number of plaques seen in different dilutions of the sample is finally used to calculate the number of infectious virus particles e.g. plaque forming units (PFU) per sample volume as a result of a plaque assay. However, the duration of the test of several days, the manual efforts required to run the test as well as the high biosafety level required prevent this test to be applied broadly in clinical routine.

In particular, SARS-CoV-2 and other beta coronaviruses have been shown to use complex molecular machinery to enter their host cell, involving proteolytic modification of the virus particle itself, which requires cellular proteases like TMPRSS2. Pharmacologic interference with these molecular processes at multiple levels in the course of recent drug development activities suggests that their completion is essential for efficient virus replication, underlining the importance of classical plaque assays to evaluate if a sample is infectious or not, and if yes, how infectious this sample actually is. Huge discrepancies have been described particularly between the results of plaque assays and RT-PCR of samples from patients with COVID-19 infections, where RT-PCR turned out to give positive results in samples that where clearly negative in classical plaque assays, indicating that non-functional non-infectious virus particles or viral RNA only might have been present in the sample.

To our surprise, investigating binding of infectious virus particles for SARS-CoV-2 to immobilized ACE2 revealed that the readout closely relates to the results obtained in classical plaque assays, while discrepancies were observed between the results of RT-PCR and the plaque assay. Moreover, using ACE2 as a detector molecule for virus particles captured by immobilized ACE2 provided best results in terms of comparability with qualitative outcomes of a plaque assay performed. From these observations we conclude that our discovery has a huge potential in replace the plaque assay in evaluating the infectious nature of a virus-containing sample paving the way for a new generation of diagnostic tests.

Using viral receptor molecules to capture and detect infectious virus particles represents a novelty with several clear advantages over the plaque assay as the closest state-of-the art. The inventive test can be realized in a self-test format, allowing high throughput broad testing without having the need for days of incubation or special biosafety measures, implying the clear potential to translate the test to clinical routine. Like the plaque assay, our approach employing virus entry receptor binding in in vitro testing is less sensitive to viral mutations, as it relies in viral functionality regarding cellular entry rather than molecular signatures like amino acid or RNA sequence.

Thus, in a particularly preferred embodiment, the virus-binding molecules comprise a virus entry receptor (for the virus particles), preferably wherein the virus entry receptor is native, recombinant, or modified recombinant.

In one embodiment of the present invention the virus-binding molecule is ACE2, preferably human ACE2, especially selected from the group consisting of native human ACE2, recombinant human ACE2, and modified recombinant human ACE2. The use of ACE2 as a virus-binding molecule is especially relevant for detection of infectious beta coronavirus particles, e.g. SARS-CoV-1 or SARS-CoV-2. It is expected that mutations occur in the viral genomes that affect RNA sequence as well as surface protein structure, which is likely to affect the reliability of tests using these signatures as their basis for readout. In case of viruses involving ACE2 or other enzymatically functional molecules in their cellular entry mechanisms, a valuable is approach is to use the soluble virus entry receptor to tag captured virus particles through binding to free spike proteins on captured virus particles. By directly measuring the enzymatic activity of the soluble virus entry receptor bound to immobilized virus particles, the detection procedure can be simplified, allowing for fluorometric or colorimetric detection of captured virus particles. Examples for virus entry receptors allowing for such an approach among coronaviruses would be ACE2 (for SARS coronaviruses and HCoV-NL63, employing ACE2) or DPP-IV (for MERS).

In one embodiment, the virus-binding molecule is human ACE2 and/or human DPP-IV, wherein it is preferably recombinant.

Importantly, using enzymatically active virus entry receptor to capture infectious virus particles on a solid phase will result in signals derived from virus-binding molecules immobilized to the solid phase. Inactivation of these virus-binding molecules is beneficial to avoid background signals, if the activity of soluble virus entry receptor molecules is used for detection of captured virus particles. The inactivation of these virus-binding molecules can be achieved by genetic, physical or chemical means, provided that the inactivation procedures do not significantly affect virus-binding.

Accordingly, in an embodiment, the inventive method may further comprise (e.g. following incubating step (a)) the steps of optionally, incubating with a washing solution, incubating with a soluble virus entry receptor for the virus particles (which may be present in an aqueous solution), preferably wherein the soluble virus entry receptor is enzymatically active, and incubating with a washing solution.

In another embodiment, the detecting step may comprise detecting soluble virus entry receptor bound to the virus particles bound to at least one virus-binding molecule bound to the solid phase, preferably detecting enzymatic activity of soluble virus entry receptor bound to the virus particles bound to at least one virus-binding molecule bound to the solid phase.

In one embodiment, the virus entry receptor bound to the solid phase is enzymatically inactive ACE2, wherein the soluble virus entry receptor is soluble enzymatically active ACE2, preferably wherein the virus particles are SARS-CoV-1, SARS-CoV-2 or HCoV-NL63 particles.

In one embodiment the virus-binding molecule is enzymatically inactive (recombinant) ACE2. In one embodiment the virus-binding molecule is genetically modified recombinant ACE2.

Enzymatically inactive recombinant ACE2 can be genetically generated by substituting individual amino acids of the enzymatic active site which is unlikely to affect its property as virus entry receptor, while efficiently removing its enzymatic activity. Alternatively, truncated versions of ACE2, still carrying the binding site for the virus might be used as a virus-binding molecule.

In one embodiment the virus-binding molecule is recombinant ACE2, which is enzymatically inactivated before incubating with the sample recombinant ACE2. Enzymatic inactivation of recombinant ACE2 can be achieved by chemical modification of the active site of the enzyme, e.g. removal of the critical Zn2+ ion by chelating agents like EDTA. Moreover, specific inhibitors of ACE2 may be used to block ACE2 activity when using ACE2 as a virus-binding molecule.

In one embodiment of the present invention, the solid phase is a planar surface made of gold, silver or metal nanoparticles.

Such surfaces are used in surface plasmon resonance (SPR) measurements, a chip technology suitable to detect adsorption of particles to a surface. Coupling virus-binding molecules (e.g. ACE2) to such a surface allows for selective binding of the virus particle to said surface, which can be detected by SPR measurement. Binding as well as dissociation kinetics for virus particles may be analyzed, allowing for a functional characterization of the virus-binding event to its virus entry receptor.

In one embodiment of the present invention, the solid phase has a semiconducting surface. The semiconducting surface may be made of different materials, which may include inorganic as well as organic semiconductors. Inorganic semiconductors include, but are not limited to silicon, germanium, gallium arsenide, silicon carbide or combinations or alloys thereof. Semiconductors may also be amorphous or liquid in nature, including but not limited to hydrogenated amorphous silicon and mixtures of arsenic, selenium and tellurium in a variety of proportions. Semiconductors may also be of organic origin, including but not limited to solids whose building blocks are pi-bonded molecules or polymers made up by carbon and hydrogen atoms and—at times—heteroatoms such as nitrogen, sulfur and oxygen. They exist in form of molecular crystals or amorphous thin films. Binding of virus particles to a virus-binding molecule immobilized to such surfaces will result in a change in conductive properties, which can be directly converted into an electronic signal.

In one embodiment of the present invention, the solid phase has a graphene surface. Graphene is an allotrope of carbon in the form of a single layer of atoms in a two-dimensional hexagonal lattice. It can also be considered as an indefinitely large aromatic molecule, the ultimate case of the family of flat polycyclic aromatic hydrocarbons. Graphene also acts as a semiconductor. Virus-binding molecules may be bound to graphene surfaces. Binding of virus particles to these virus-binding molecules would result in a change in electronic properties of the graphene layer, which can be converted into a signal that can be directly measured and reflects the amount of virus particles bound to the surface. Graphene is a preferred material to realize a routine lab-on-chip solution. Virus-binding molecules (e.g. ACE2) may be covalently linked to the graphene carbon atoms, providing a very stable material suitable to capture virus particles, which would have advantages in the production and commercialization of a test.

In one embodiment of this invention, a new test for the detection of SARS-CoV-2 in saliva or aerosol samples is provided, which is based on the use of angiotensin converting enzyme 2 (ACE2) as the known cellular input receptor for this virus. Instead of ACE2 also related molecules with comparable binding properties for the virus can be used.

The novel idea to utilize the cellular receptor for virus entry offers a decisive advantage over known classic test methods:

The infectious potential of a virus depends on several factors, with binding to the cellular receptor of the host for virus entry being a key property. As with SARS-CoV-1, for SARS-CoV-2 the cellular receptor is ACE2, a carboxypeptidase of the renin-angiotensin-aldosterone system (RAAS), which is expressed on the surface on type II alveolar cells. This receptor serves as a virus entry point for infection of the lungs. However, ACE2 is also expressed on a variety of other cells of the host indicating the threat of infection not only of the lungs but subsequently also of other vital organs and tissues of the infected individual.

For evolutionary reasons, the binding between virus and ACE2 is an interaction with high affinity. The responsible protein on the virus is the so called “Spike Protein 2” (the “S protein”). The high affinity of the S protein for its cognate ACE2 receptor is important for the virus to efficiently initiate the infection process. A key element of aspects of the invention is the utilization for a novel test system of the high affinity and specificity of binding of SARS-CoV-2 via its S protein to its cellular entry receptor ACE2. As long as the virus—despite potential mutations—uses ACE2 for cellular entry, assay formats described herein will detect it. This is in striking difference to nucleic acid-based tests, such as rRT-PCR based detection with specific primers for which virus mutations may cause problems.

The test principle of this invention may include the immobilization of recombinant ACE2 on a solid phase, which then is brought into contact with the sample material in order to capture and bind the virus particles with high efficiency (=>“Capture-ACE2”).

Instead of recombinant ACE2 also other molecules with comparable binding affinity and specificity can be utilized. In one embodiment of the present invention, this molecule is enzymatically inactive recombinant ACE2, which is described in literature and is for instance obtained by exchanging two amino acids in the region responsible for Zn²⁺ binding, a cofactor for ACE2 activity. Thereby the affinity to Zn²⁺ is lost leading to an enzymatically inactive protein which however retains the same binding affinity to the S protein of SARS-CoV-2.

There are a variety of ways to immobilize recombinant proteins on solid phases. Different linkers, tags and carrier materials can be used in various combinations. In addition to immobilization, an appropriate buffer composition may also be an important aspect of the test system.

Captured virus can be detected by a variety of methods. In one embodiment of this invention the virus is captured by inactivated recombinant ACE2 and detection is based on saturation of free S protein molecules on the surface of bound virus particles with soluble recombinant ACE2, which then is detected directly via a measurement based on ACE2 enzymatic activity to cleave certain peptide substrates. This method has an intrinsic amplification effect, as multiple binding sites for soluble virus entry receptors are available on the surface of a virus particle. The ACE2 activity can be measured using a variety of methods: LC-MS/MS based methods for activity determination using high affinity natural substrates, fluorometric methods or colorimetric methods can be used. The use of protein purification, affinity and/or fluorescence tags could also be used to detect soluble virus entry receptor bound to captured virus particles. Many viruses enter the cells through enzymatically active cellular entry receptors, allowing for easy extension of the described approach. Using the interaction of virus in capture and detection steps may provide additional information on the infectious state of an infectious sample, as shown in EXAMPLE 1 below.

In another embodiment of the present invention, captured virus is detected by direct chip-based technologies, which can sense the forces a virus imposes onto the solid phase capture matrix upon binding of virus particles. In another embodiment of the present invention, surface plasmon resonance technology may be used to directly detect virus bound to a solid phase capture matrix presenting cellular virus entry receptors.

In another embodiment of this invention, multiple different capture molecules may be immobilized onto the same carrier allowing the simultaneous capture of multiple different viruses on the solid phase. Specific detection may be performed using a cocktail of different fluorescent markers or nanoparticle-labeled antibodies for virus detection and identification.

In another embodiment of this invention, virus captured by recombinant ACE2 on the solid phase is detected by appropriate antibodies to the S protein or other surface molecules and fluorometric or colorimetric reporting as usually applied in ELISA and related systems. Using multiple different capture molecules would allow for different virus strains to be simultaneously captured from a sample.

In another embodiment of this invention, combinations of antibodies linked to different detection systems may be used to simultaneously and specifically detect multiple viruses using the same entry receptor or capture molecule.

In another embodiment of the present invention, multiplex detection of different viruses is simultaneously performed, by using multiple antibodies that are specifically labeled with fluorescence tags, nano-particles or enzymatic labels sufficient to distinguish different analytes.

In another embodiment of this invention, for precise characterization of the captured virus by the virus entry receptor ACE2 or a variant thereof, RT-PCR is applied. Such characterization may be desired to verify that SARS-CoV-2 has been captured. It is known that a certain corona virus that causes respiratory tract infections, i.e. common cold, named HCoV-NL63, also uses ACE2 expressed on host cells for viral entry. Therefore, HCoV-NL63 may be also captured and detected as described above, although the affinity of the S protein of HCoV-NL63 is different compared to that of the S protein of SARS-CoV-2. By using RT-PCR with probes specific for SARS-CoV-2, a precise differentiation to HCoV-NL63 is achieved. On the other side, HCoV-NL63 can be identified in such a VERB test by using a HCoV-NL63-specific probe for the RT-PCR with the captured virus.

Similarly, SARS-CoV-1, which also uses ACE2 as virus entry receptor, can be detected by the described VERB-test principle in certain embodiments. Using specific probes for this virus, a differentiation to SARS-CoV-2 can be achieved easily.

In another embodiment of the present invention, PCR is employed to detect captured virus. As for antibodies, multiplexing applications are possible using this approach. Although PCR detection could be affected my virus mutations, PCR offers a high sensitivity on top of the information gained by combining this technology with the virus entry receptor capture technology descried in the present invention (EXAMPLE 1).

The method described in the present invention is compatible with a variety of different sample material.

In one embodiment of the present invention, the sample may be a clinical sample (i.e. a sample obtained from a patient), preferably comprising bronchoalveolar lavage (BAL) fluid, sputum, tracheal aspirate, epithelial cells in particular obtained by an epithelial swab such as an nasopharyngeal swab. Samples may e.g. be diluted in an appropriate binding buffer, before performing the binding step to our solid-phase capture matrix. The dilution or binding buffer should preserve viral structural integrity, to lead to a higher signal in our method. Other commonly used clinical sampling procedures that collect swabs from epithelial surfaces (nose, pharynx, genital, anal) may also be used in our method following resuspension in an appropriate dilution buffer. The same principle is applicable for non biological surfaces.

In another embodiment of the present invention, the sample is a body fluid such as blood, serum, plasma or urine. Although blood titers of SARS-CoV-2 are very low in humans, combination of the VERB-Assay with PCR allows for detection of infectious virus particles in blood based samples. (EXAMPLE 1)

In another embodiment of this invention, the process of capturing virus via S protein and its entry receptor ACE2 can be used to analyze the neutralizing potency of serum or plasma samples of an individual who recovered from a previous SARS-CoV-2 infection. If a serum or plasma sample contains neutralizing antibodies, binding of SARS-CoV-2 virus particles, or preferred inactivated SARS-CoV-2 virus particles to inactivated recombinant ACE2 on the matrix will be inhibited. Inactivation can be performed by heat or other known methods that only minimally alter the structure of virus proteins. Titrating various amounts of immune serum or plasma into a suspension containing inactivated SARS-CoV-2 virus particles in such a VERB-test format therefore allows to estimate the neutralizing potency of induced antibodies in such sample by determining the concentration which is needed to get e.g. 50% inhibition of binding of inactivated SARS-CoV-2 virus to inactivated recombinant ACE2.

In another embodiment of the present invention, the immobilization of virus particles by binding to a solid phase capture matrix is performed in the presence of body fluids, preferably blood, plasma, serum or epithelial swabs. Neutralizing antibodies reduce our signals obtained from analyzing a sample containing infectious virus (EXAMPLE 2) defined, preferably inactivated virus preparation.

In another embodiment of the present invention, such an inactivated virus preparation is produced by mild inactivation procedures preferentially targeting viral RNA or DNA, maintaining surface structures and receptor binding properties of virus particles.

In another embodiment of the present invention, the sample is an aerosol. Aerosols are very small liquid droplets that can contain infectious virus. Human-to-human transmission of respiratory viruses including SARS-CoV-2 occurs primarily via droplet infection, which involves aerosols generated during speaking, but primarily with infection symptoms like coughing or sneezing. Given that respiratory viruses replicate within the lungs, the concentration of virus particles in lung-derived aerosols may be very high.

In another embodiment of the present invention, a solid phase capture matrix coated with immobilized viral entry receptor may be used to capture infectious virus particles from air streams, which could be exhaled air or air collected from air supply systems in airplanes, buses or other means of public transport. Such measures could significantly contribute to prevent the spread of pandemic viral infections.

Methods described herein may be the basis for diagnostic kits to detect virus infections and also to detect neutralizing antibodies in patients that already went through a viral infection. The principle may be applied to a variety of different viruses and could set a new standard to monitor anti-viral vaccination efforts and detect infectious patients in a pandemic setting. Such kits may comprise a manual, one or more solvents, one or more buffers, and/or one or more solid phases and/or one or more enzymes and/or one or more antibodies and/or one or more primers and/or one or more enzyme substrates and/or one or more inactivated virus preparations.

The present invention is further explained by the following figures and examples, without being restricted thereto.

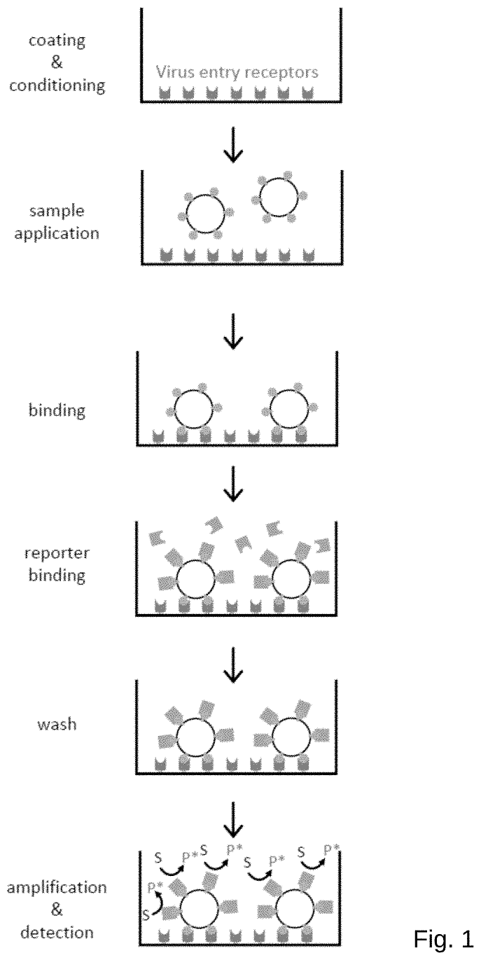

In FIG. 1, the assay principle based on capturing virus particles with a molecule resembling the virus's receptor for entry into human cells (e.g., inactivated recombinant ACE2) and detecting virus particles using the enzymatic activity of recombinant ACE2 as reporter is schematically visualized (virus entry receptor binding test, VERB test). In the figure, the principle of a VERB assay using a microtiter plate coated with a virus-binding molecule (red) is shown. The scheme describes detection using the activity of soluble virus entry receptor molecules (green) inducing a fluorescence-, color- or chemiluminescence-generating substrate/product conversion.

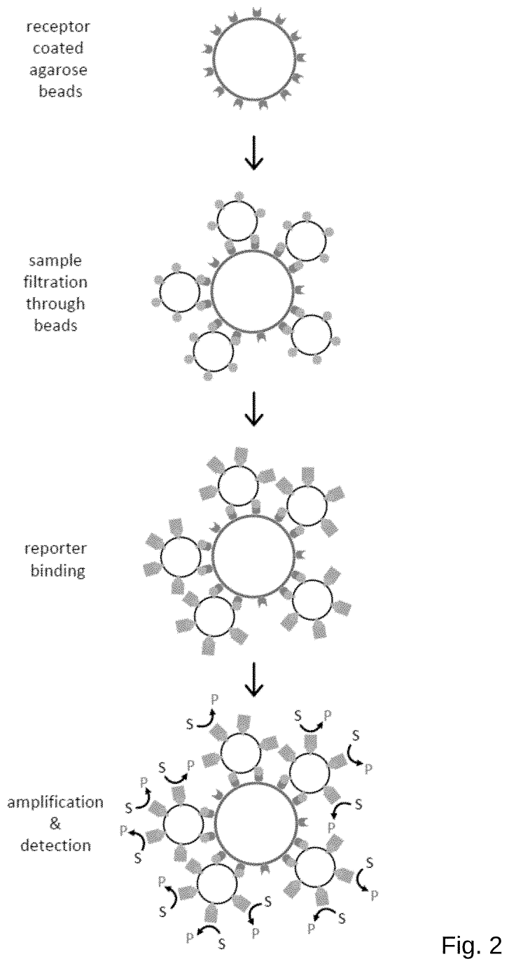

In another embodiment of this invention, an alternative technical setup is described, which enables higher sample volumes. FIG. 2 shows an assay principle based on capture with inactivated recombinant ACE2 bound to beads. This leads to a significant improvement in the surface/volume ratio and thus to a more efficient binding of virus particles from the sample. Furthermore, this principle can be carried out as liquid extraction column or even aerosol test to analyze respiratory secretions, saliva, breathing air, but also plasma or urine or other body fluids. Based on this assay principle, a chromogenic point-of-care test is feasible, that reports the presence of infectious virus particles as a color change within a few minutes. In the figure, the principle of a VERB assay using agarose beads coated with a virus-binding molecule (red) is shown. The scheme describes detection using the activity of soluble virus entry receptor molecules (green) inducing a fluorescence-, color- or chemiluminescence-generating substrate/product conversion. Different types of beads could be used as a capture matrix, including but not limited to agarose beads, magnetic beads or luminex beads.

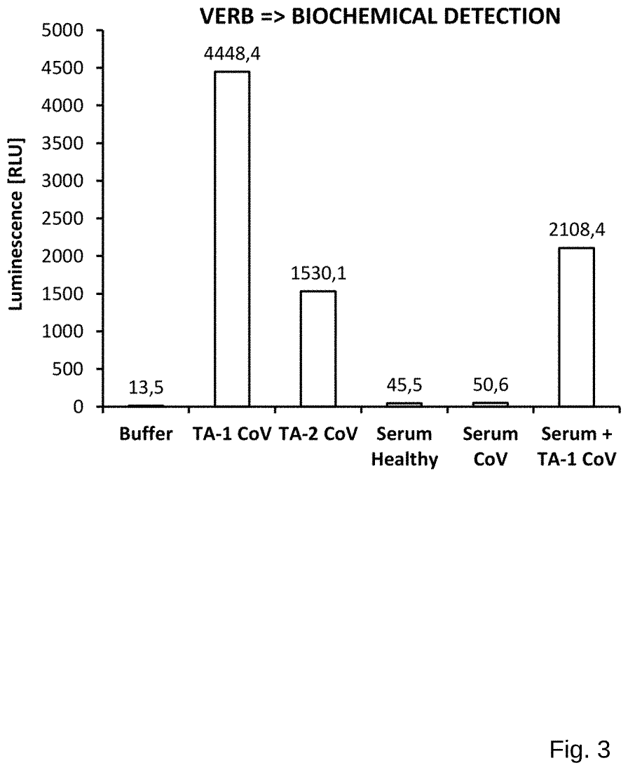

FIG. 3: Comparative analysis of COVID-19 VERB results for tracheal aspirate (TA) and serum from two COVID-19 (CoV) patients, also investigating matrix (Serum+TA-1 CoV) using detection of viral entry receptor as biochemical readout (HRP substrate).

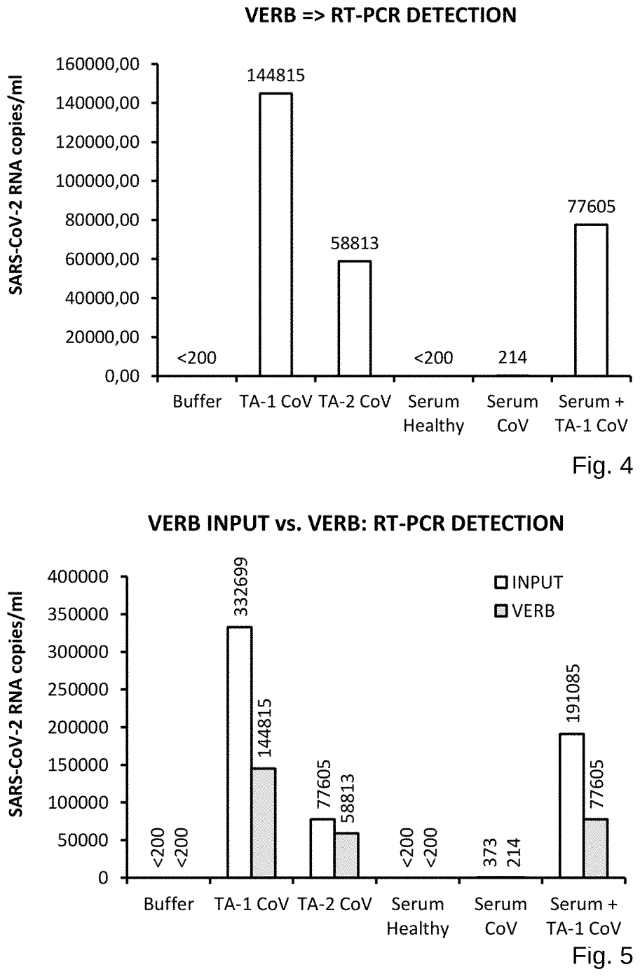

FIG. 4: Comparative analysis of COVID-19 VERB results for tracheal aspirate (TA) and serum from two COVID-19 (CoV) patients, also investigating matrix (Serum+TA-1 CoV) using RT-PCR based detection.

FIG. 5: Comparing the results obtained from RT-PCR analysis of the VERB input with the VERB result following RT-PCR detection.

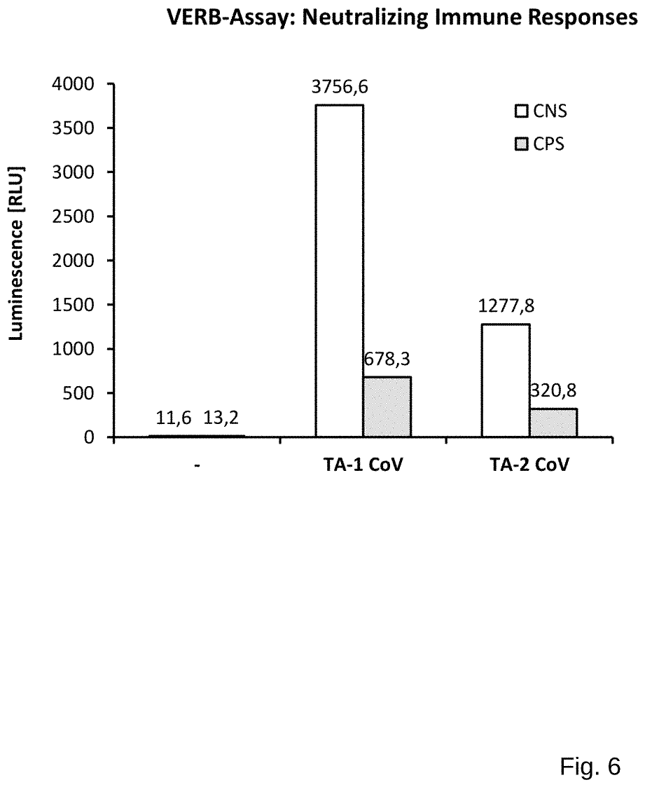

FIG. 6: Effects of 1% serum from COVID-19 negative (CNS) and COVID-19 positive convalescent serum (CPS) on VERB signals obtained from tracheal aspirate of a COVID-19 patient.

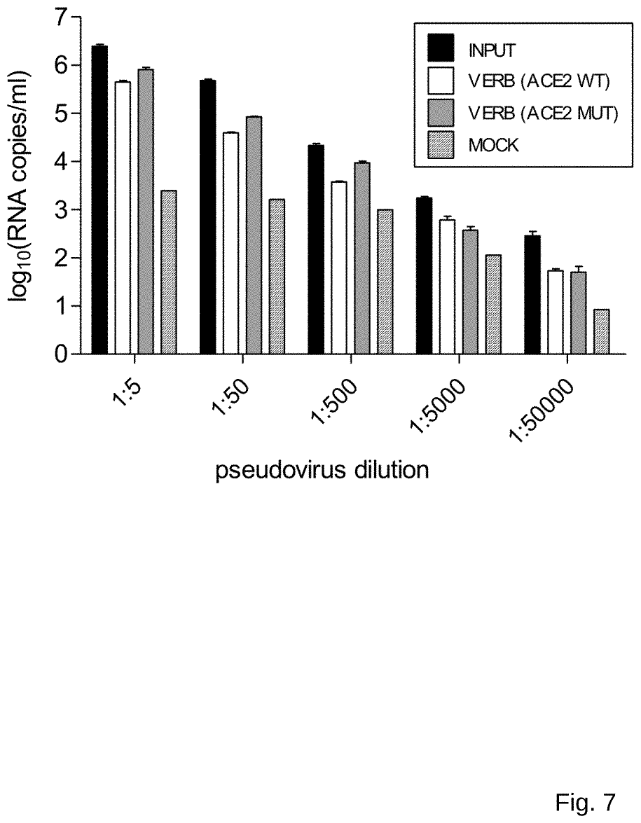

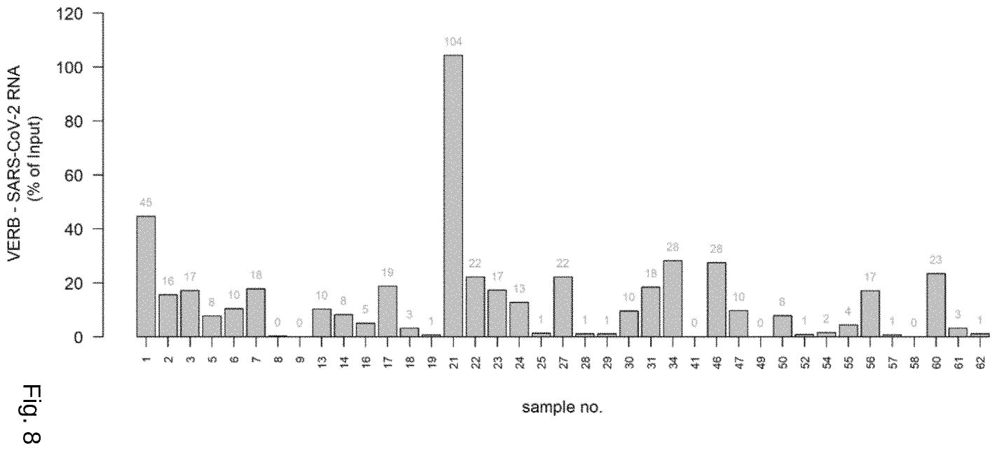

FIG. 7: VERB capture of a dilution series of retrovirus pseudotyped with SARS-CoV-2 spike protein using RT-PCR based detection.

FIG. 8: Fraction of SARS-CoV-2 RNA (% of Input) obtained after VERB capture from nasopharyngeal swab samples from COVID-19 patients.

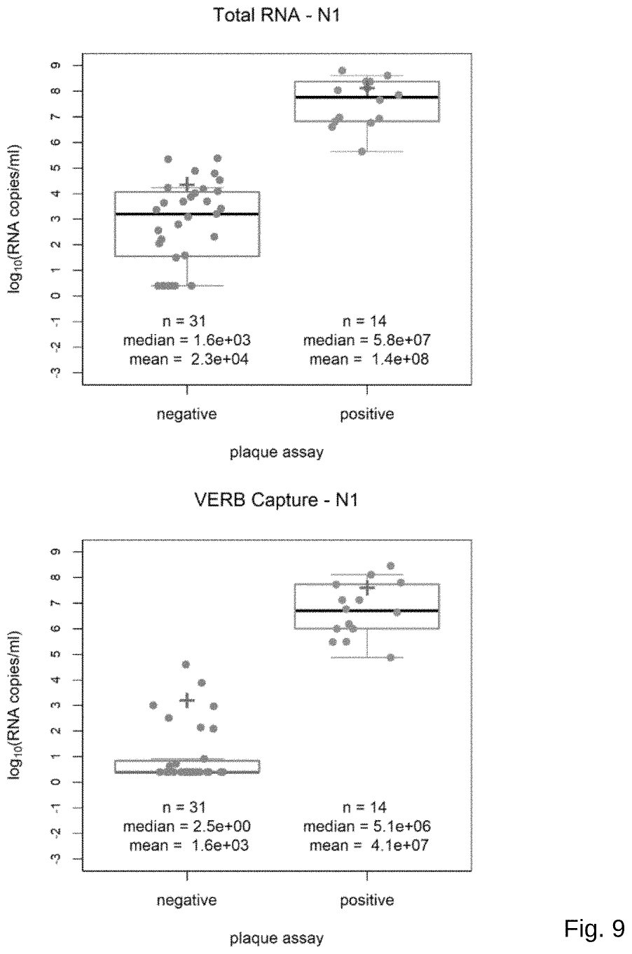

FIG. 9: Correlation of infectivity in classical plaque assay to RT-qPCR analysis from Input (Total RNA) and VERB Capture in nasopharyngeal swab samples from COVID-19 patients. Boxes represent the interquartile range; horizontal lines indicate median values; “+” indicate mean values.

### Example 1: Binding of SARS-CoV-2 Particles to Solid Phase Capture Matrix Coated with rhACE2

The capture of SARS-CoV-2 was performed with His-tagged recombinant human ACE2. Samples used included tracheal aspirate (TA) and serum from SARS-CoV-2 infected patients. We used Ni-NTA plates (96-well) as a solid phase to immobilize His-tagged ACE2 for virus capture. ACE2 coated plates were incubated with pretreated tracheal aspirate or serum samples as indicated, followed by multiple washing steps. Following virus-binding, wells were incubated with soluble biotin labeled recombinant human ACE2 to saturate free ACE2 binding sites on virus particles, followed by multiple washing steps. For detection, we used streptavidin labeled HRP in combination with a high sensitivity chemiluminescence substrate (FIG. 3). Luminescence signals (RLU) were detected in all samples containing tracheal aspirate. Tracheal aspirate diluted 1:1 in serum from a healthy volunteer resulted in a reduced signal corresponding to the dilution factor employed. Following biochemical detection, sample were subjected to RT-PCR for determination of virus titers in VERB wells (FIG. 4). Signals for RT-PCR based detection and biochemical detection of analyzed samples were highly correlating. Given that biochemical detection requires the presence of multivalent binding sites on captured particles, we conclude that intact virus can be efficiently captured on solid phase capture matrix. To our surprise, comparing the VERB input with the VERB output using RT-PCR revealed that the ratio between output and input was significantly different between the two individual patients analyzed, suggesting the presence of large amounts of viral RNA in patient 1 (TA-1), that is not associated with intact virus particles and could therefore not be detected in the VERB assay (FIG. 5). Importantly, the ratio between TA-2 and TA-1 for results obtained in a classical cellular plaque assay for COVID-19 was comparable to the ratios obtained from VERB assays using either RT-PCR or biochemical detection of the viral entry receptor (Table 1, below), while the RT-PCR based determination of titers in input samples revealed a clearly different ratio between TA-2 and TA-1, suggesting that the outcome of the VERB assay reflects classical cellular plaque assays. We conclude, that the VERB assay can be used to discriminate between intact and infectious virus particles and disintegrated virus fragments, still leading to (false positive) signals in RT-PCR based analysis of clinical samples, as shown for tracheal aspirates.

TABLE 1
Direct comparison of plaque assay with RT-PCR and VERB detection
in tracheal aspirates of two COVID-19 patients.

| | INPUT | | VERB | |
|---|---|---|---|---|
| | RT-PCR | Plaque Assay | RT-PCR | ACE2 |
| | [copies/ml] | [PFU/ml] | [copies/ml] | [RLU] |
| TA-1 CoV | 332699 | 1.4E+05 | 144815 | 4448.4 |
| TA-2 CoV | 77605 | 4.9E+04 | 58813 | 1530.1 |
| TA-2/TA-1 | 23% | 36% | 41% | 34% |

### Example 2: Monitoring Neutralizing Antibodies

In another experiment, we performed the binding step for tracheal aspirates in the presence of serum. Serum from healthy volunteers (CNS, COVID-19 negative serum) and serum from convalescent COVID-19 patients (CPS, COVID-19 positive serum) was used at a dilution of 1:100 during virus capture. Serum was mixed with the virus containing samples (TA-1 CoV and TA-2 CoV) and incubated for 60 min before incubating with His-tag ACE2 coated Ni-NTA plates. Following binding and washing as described before, chemiluminescence signals were analyzed. The presence of serum from convalescent COVID-19 patients selectively resulted in a profound suppression of obtained VERB signals, suggesting the inhibition of the interaction between plate immobilized virus entry receptor and virus particles. Therefore we conclude that a modified setup of the VERB assay is suitable to detect neutralizing antibodies against COVID-19 in serum samples.

### Example 3: Binding of Spike-Pseudotyped Retroviral Particles to Magnetic Beads Coated with ACE2 or Enzymatically Inactive ACE2

The capture of retroviruses pseudotyped with SARS-CoV-2 Spike protein was performed with magnetic beads coated either with enzymatically active ACE2 or an enzymatically inactive mutant of ACE2. Specifically, streptavidin magnetic beads were used as a solid phase to immobilize biotinylated ACE2 for virus capture. In addition, non-coated beads (“MOCK”) were included as a control for unspecific binding of particles to the solid phase. ACE2-coated beads were incubated with a dilution series of pseudotyped retrovirus in phosphate buffered saline, followed by multiple washing steps. For detection, the beads were incubated in RNA lysis buffer and subsequently, RNA was extracted using a commercially available kit. VERB captured RNA in comparison to total RNA extracted from input samples was quantified by RT-qPCR (see FIG. 7). Captured RNA (VERB) clearly correlated with total RNA (Input) in a dose-dependent manner, when using ACE2-coated beads. In contrast, significantly less RNA was detected after incubation with uncoated beads (Mock), thus suggesting that the capture of RNA from a Spike-pseudotype virus preparation is specific for ACE2. Surprisingly, a linear relationship of input to captured RNA was observed over the entire dilution series, covering five orders of magnitude. In addition, equal capture efficiencies were obtained by using an enzymatically inactive mutant of ACE2. We conclude that VERB capture is highly efficient, works in a quantitative and dose-dependent manner over a wide range of virus concentrations and is independent of the enzymatic activity of ACE2.

### Example 4: A Semi-Automated VERB Capture Platform for the Analysis of Clinical Samples

The capture of SARS-CoV-2 from nasopharyngeal swab (NPS) samples was performed using biotinylated recombinant human ACE2 immobilized on streptavidin magnetic beads. To compare VERB captured SARS-CoV-2 to total RNA (input) from unprocessed samples, samples from different patients were each diluted 1:2 in saline and each of the samples was divided into two equal parts. The first part of each sample was used for direct RNA extraction (called “Input” in FIG. 8), whereas the second part of each sample was subjected to VERB capture, followed by RNA extraction. Briefly, samples were incubated with VERB beads for 30 minutes at room temperature with constant agitation, followed by two wash steps in phosphate buffered saline using an automated magnetic handler (TANbead Maelstrom 8). Subsequently, beads were incubated in RNA lysis buffer and RNA from Input samples and VERB capture samples, were extracted in parallel. We analysed the RNA content of the samples by RT-qPCR using a primer-probe set targeting the nucleocapsid gene of SARS-CoV-2.

Surprisingly, the fraction of captured virus varied dramatically between different patients (see FIG. 8), which suggests that classical RT-qPCR analysis does not allow a proper estimate of the amount of infectious virus present in clinical samples.

To directly establish the link to infectivity, a subset of samples was examined in a classical cellular plaque assay (see also FIG. 9). Already in input samples (total RNA), a difference RNA copies/ml was apparent in samples with a positive signal in plaque assay compared to samples not yielding any plaques (negative). Although the ratio of median RNA copies/ml for positive samples (median=5.8e7) to the median RNA copies/ml of negative samples (median=1.6e3) was already more than 10,000-fold, this difference became even more pronounced after VERB capture. Plaque assay positive samples still showed median RNA copies/ml of 5.1e6, whereas negative samples were mostly also negative in RT-qPCR, resulting in a median RNA copies/ml of 2.5. In summary, we conclude that results obtained after VERB capture are a good predictor of infectivity and correlate well with a classical plaque assay.

1. A method for detecting virus particles in a sample, comprising the steps of:
(a) incubating the sample with at least one virus-binding molecule bound to a solid phase; and
(b) detecting binding of virus particles to the at least one virus-binding molecule bound to the solid phase.

2. The method of claim 1, wherein the virus-binding molecules comprise a virus entry receptor.

3. The method of claim 1, wherein the at least one virus-binding molecule comprises angiotensin-converting enzyme 2 (ACE2), selected from the group consisting of native human ACE2, recombinant human ACE2, and modified recombinant human ACE2; wherein the virus particles are severe acute respiratory syndrome coronavirus (SARS-CoV)-1 particles, SARS-CoV-2 particles or HCoV-NL63 particles.

4. The method of claim 1, wherein the sample is a clinical sample, comprising bronchoalveolar lavage (BAL) fluid, sputum, tracheal aspirate, epithelial cells obtained by an epithelial swab, or a body fluid such as blood, serum, plasma or urine.

5. The method of claim 1, wherein the binding is detected directly or indirectly.

6. The method of claim 1, wherein the binding is detected by one or more labelled antibodies.

7. The method of claim 1, wherein the binding is detected by one or more soluble recombinant virus entry receptors.

8. The method of claim 1, wherein the binding is detected by PCR.

9. The method of claim 1, wherein the sample is an aerosol.

10. The method of claim 1, wherein the sample is a native or inactivated virus or pseudo virus preparation.

11. The method of claim 1, wherein the sample is incubated in the presence of body fluids.

12. The method of claim 1, wherein the sample is incubated in the presence of neutralizing antibodies.

13. The method of claim 1, wherein the virus particles are infectious virus particles.

14. The method of claim 1, wherein the at least one virus-binding molecule bound to the solid phase comprises a virus entry receptor bound to the solid phase, wherein the virus entry receptor bound to the solid phase is enzymatically inactive.

15. The method of claim 1, further comprising the steps of:
incubating with a washing solution;
incubating with a soluble virus entry receptor for the virus particles; and
incubating with a washing solution.

16. The method of claim 1, wherein said detecting step (b) comprises detecting soluble virus entry receptor bound to the virus particles bound to at least one virus-binding molecule bound to the solid phase.

17. The method of claim 1, wherein the virus entry receptor bound to the solid phase is enzymatically inactive ACE2, wherein the soluble virus entry receptor is soluble enzymatically active ACE2, wherein the virus particles are SARS-CoV-1 particles, SARS-CoV-2 particles or HCoV-NL63 particles.

18. The method of claim 1, wherein the solid phase is a plate such as a 96-well plate, beads such as agarose beads or magnetic beads, a solid phase having a graphene surface or a solid phase having a semiconducting surface.

19. A kit for performing the method of claim 1, comprising a manual, one or more solvents, one or more buffers, and/or one or more solid phases and/or one or more enzymes and/or one or more antibodies and/or one or more primers and/or one or more enzyme substrates and/or one or more inactivated virus or pseudo virus preparations.

20. A kit for detecting virus particles in a sample, preferably for use in the method of claim 14, comprising:
a solid phase being one of a plate, beads such as agarose beads, a solid phase having a graphene surface, and a solid phase having a semiconducting surface;
a virus entry receptor for the virus particles bound to the solid phase;
a soluble virus entry receptor for the virus particles;
at least one substrate for the enzymatic activity of the soluble virus entry receptor;
at least one washing solution; and
at least one inactivated virus or pseudo virus preparations;
wherein:
the virus entry receptor bound to the solid phase is enzymatically inactive ACE2, the soluble virus entry receptor is soluble enzymatically active ACE2, wherein the virus particles are SARS-CoV particles, SARS-CoV-2 particles or HCoV-NL63 particles.
